(19) United States
(12) Patent Application Publication
(10) Pub. No.: US 2008/0027079 A1
(43) Pub. Date: Jan. 31, 2008

(54) DIHYDROOROTATE DEHYDROGENASE INHIBITORS WITH SELECTIVE ANTI-MALARIAL ACTIVITY

(75) Inventors: Margaret Phillips, Dallas, TX (US); Pradipsinh K. Rathod, Seattle, WA (US); Jeffery Baldwin, Dallas, TX (US); Ramesh Gujjar, Seattle, WA (US)

Correspondence Address: FOLEY AND LARDNER LLP;SUITE 500, 3000 K STREET NW, WASHINGTON, DC, 20007, US

(73) Assignees: Board of Regents, University of Texas System; University of Washington

(21) Appl. No.: 11/758,137

(22) Filed: Jun. 5, 2007

Related U.S. Application Data
(60) Provisional application No. 60/815,568, filed on Jun. 22, 2006.

(51) Int. Cl. A61K 31/519; A61P 43/00

(52) U.S. Cl. 514/259.31

(57) ABSTRACT

Pharmaceutical compositions comprising compounds of the formula 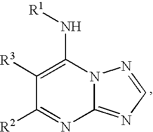
where R¹, R², and R³ are described here, have therapeutic utility in selectively inhibiting P. falciparum dihydroorotate dehydrogenase. Accordingly, such compositions have use in the treatment and prevention of malaria.

### CROSS-REFERENCE TO RELATED APPLICATIONS

This application claims the benefit of priority to U.S. Provisional Patent Application No. 60/815,568, which was filed on Jun. 22, 2006.

### GOVERNMENT RIGHTS

This invention was funded NIH R01 A1053680. The U.S. Government has certain rights in this invention.

### BACKGROUND OF THE INVENTION

The present invention relates generally to novel anti-malarial agents and inhibitors of dihydroorotate dehydrogenase.

Malaria infects up to 900 million people and causes as many as 2.7 million deaths worldwide every year. Nearly 40% of the world population is at risk for contracting this disease, which has been a major cause of mortality throughout history. In the United States travelers to these endemic regions are at risk for contracting the disease. The widespread emergence of drug resistance in many tropical countries has compromised many of the current chemotherapies and there is a continued need for new chemotherapeutic approaches.

Malaria is a disease caused by a parasite transmitted by the bite of an infected female Anopheles mosquito. When an infecting sporozoite parasite enters the bloodstream it rapidly infects both liver and red blood cells and differentiates into merozoites. Asexual reproduction of the merozoite within erythrocytes results in the rupture and subsequent reinfection of other red blood cells. This cyclic process results in clinical symptoms, which include headaches, sweating, vomiting, malaise, delirium and acute fever and may be fatal if not treated. Malaria in humans is caused by 4 species of parasitic protozoa belonging to the genus Plasmodium. Of these, P. falciparum is the most deadly and the greatest threat to travelers abroad while P. malariae, P. vivax and P. ovale, though infrequently fatal in healthy adults, can cause morbidity in the endemic areas.

Various medications are presently used for the treatment of malaria. However, many of these medications are costly and some exhibit significant toxicity and undesirable side effects in humans. The most common drug for treating malaria is chloroquine. Other drugs include quinine, melfloquine, atovaquone/proguanil, doxycycline, artesunate, hydroxychloroquine, halofantrine, pyrimethamine-sulfadoxine, and primaquine. Drug choice often depends on one of the four types of malaria parasites.

Malaria parasites rely on de novo pyrimidine biosynthesis to provide precursors for DNA and RNA synthesis, hence for proliferation. The parasite does not have pyrimidine nucleoside or base salvage pathways, thus the enzymes in the de novo pathway are essential to parasite survival. In contrast, mammalian cells have salvage pathways that provide an alternative route to these essential metabolites.

Dihydroorotate dehydrogenase (DHODH) is an essential enzyme for the salvage pathway, and a number of lines of evidence suggest that it is an important target for the development of new chemotherapy against malaria. DHODH is a flavin-dependent mitochondrial enzyme that catalyzes the fourth reaction in the salvage pathway; coenzyme Q is utilized as the oxidant. The enzyme has a number of properties that make it a particularly strong candidate as a new drug target in the parasite. Inhibitors of human DHODH have proven efficacy for the treatment of rheumatoid arthritis demonstrating that the target pathway can be effectively blocked in vivo. The X-ray structures of DHODH reveal that the inhibitor binding pocket of the enzyme is highly variable between species, providing a structural basis for the design of species-specific inhibitors.

A need exists for a method of treating malaria. There is also a need for an anti-malarial agent to overcome current drug resistance problems with existing therapy. Further, anti-malarial agents are needed that selectively inhibit malarial DHODH but exhibit no substantial toxicity against mammalian, especially human DHODH.

Accordingly, this invention provides novel potent anti-malarial agents and methodology of treating malaria using novel potent anti-malarial agents. The invention also provides potent anti-malarial agents that are selective inhibitors of P. falciparum dihydroorotate dehydrogenase and active against chloroquine-sensitive and resistant malarial strains.

### SUMMARY OF THE INVENTION

The present invention relates to novel pharmaceutical compositions for inhibiting the activity of Plasmodium falciparum dihydroorotate dehydrogenase. The novel pharmaceutical compositions display selective inhibition of Plasmodium falciparum dihydroorotate dehydrogenase over human dihydroorotate dehydrogenase.

The present invention also relates to methods for preventing or treating diseases associated with the action of Plasmodium falciparum dihydroorotate dehydrogenase, such as malaria.

The pharmaceutical compositions of the inventions are pharmaceutical compositions comprising a compound of the formula 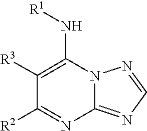
or pharmaceutically acceptable salts, solvates, stereoisomers, tautomers, or prodrugs thereof, and a pharmaceutically acceptable carrier.

R¹ is selected from the group consisting of (C₈-C₁₄) heterocycloalkyl, aryl, and heteroaryl, where the heterocycloalkyl, aryl or heteroaryl has two or more rings.

Each of R² and R³ is selected from the group consisting of halogen, (C₁-C₈)alkyl, (C₂-C₈)alkenyl, (C₂-C₈)alkynyl, (C₁-C₈)alkoxy, and (C₁-C₈)haloalkyl.

Any heterocycloalkyl, aryl or heteroaryl is optionally substituted with one or more members selected from the group consisting of halogen, —CN, —NO₂, hydroxyl, (C₁-C₈)alkyl, (C₂-C₈)alkenyl, (C₂-C₈)alkynyl, (C₁-C₄)alkoxy, (C₁-C₄)haloalkyl, and (C₂-C₄)hydroxyalkyl.

In another embodiment, the pharmaceutical composition of the invention arcomprises a compound of formula (I) 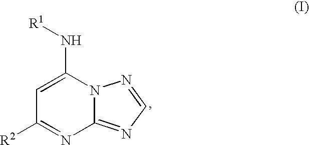
or pharmaceutically acceptable salts, solvates, stereoisomers, tautomers, or prodrugs thereof, and a pharmaceutically acceptable carrier. Variables R¹ and R² are as defined above.

In one embodiment, optionally in combination with any other embodiment herein described, the invention provides methods for treatment of malaria, comprising administering to a patient in need thereof a therapeutically effective amount of a pharmaceutical composition comprising a compound as herein defined.

In another embodiment, optionally in combination with any other embodiment herein described, the invention provides methods of inhibiting dihydroororate dehydrogenase in a parasite, comprising contacting said parasite with a pharmaceutical composition of comprising a compound as herein defined.

In yet another embodiment, optionally in combination with any other embodiment herein described, the invention provides methods of inhibiting dihydroororate dehydrogenase of a malaria parasite in a host mammal, comprising administering to the host mammal an effective amount of a pharmaceutical composition comprising a compound as herein defined, whereby mammalian dihydroororate dehydrogenase is not inhibited.

In one embodiment, optionally in combination with any other embodiment herein described, optionally in combination with any other embodiment herein described,the invention provides methods of killing a Plasmodium falciparum parasite comprising contacting said parasite with an effective amount of a pharmaceutical composition comprising a compound as herein defined.

In another embodiment, optionally in combination with any other embodiment herein described, the invention provides methods of killing Plasmodium falciparum parasites in a host mammal comprising administering to the host mammal in need thereof a therapeutically effective amount of a pharmaceutical composition comprising a compound as herein defined.

These and other embodiments of this invention will be evident upon reference to the following detailed description. To that end, certain patent and other documents are cited herein to more specifically set forth various embodiments of this invention.

### BRIEF DESCRIPTION OF THE DRAWINGS

FIG. 1 shows selective inhibition of the P. falciparum dihydroorotate dehydrogenase enzyme by a compound of formula I. By contrast, there is an absence of activity in evidence against human dihydroorotate dehydrogenase (hDHODH).

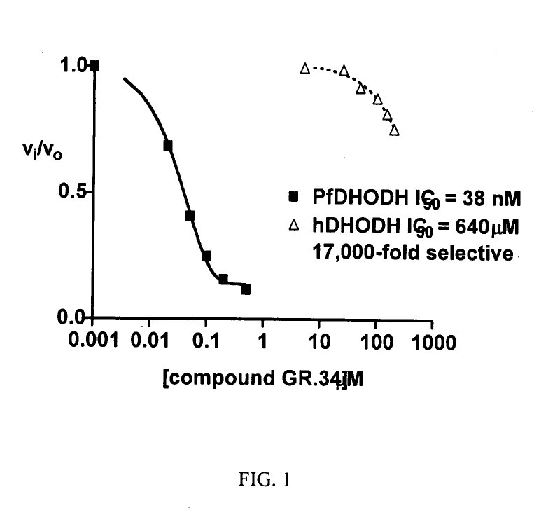

FIG. 2 is a graph that depicts activity of a compound of formula I against cultured P. falciparum malarial parasites. Results are from a whole cell assay. In comparison, the compound is non-toxic in a cultured mammalian cell line.

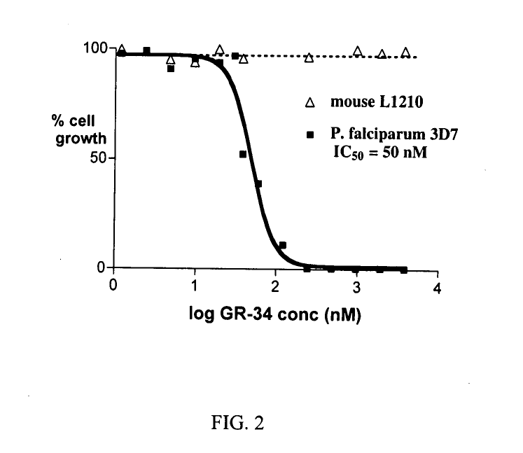

### DETAILED DESCRIPTION OF THE INVENTION

Definitions

The term “alkyl” refers to a straight or branched chain, saturated hydrocarbon having the indicated number of carbon atoms. For example, (C₁-C₆)alkyl is meant to include but is not limited to methyl, ethyl, propyl, isopropyl, butyl, sec-butyl, tert-butyl, pentyl, isopentyl, neopentyl, hexyl, isohexyl, and neohexyl. An alkyl group can be unsubstituted or optionally substituted with one or more substituents as described herein below.

The term “alkenyl” refers to a straight or branched chain unsaturated hydrocarbon having the indicated number of carbon atoms and at least one double bond. Examples of a (C₂-C₈)alkenyl group include, but are not limited to, ethylene, propylene, 1-butylene, 2-butylene, isobutylene, sec-butylene, 1-pentene, 2-pentene, isopentene, 1-hexene, 2-hexene, 3-hexene, isohexene, 1-heptene, 2-heptene, 3-heptene, isoheptene, 1-octene, 2-octene, 3-octene, 4-octene, and isooctene. An alkenyl group can be unsubstituted or optionally substituted with one or more substituents as described herein below.

The term “alkynyl” refers to a straight or branched chain unsaturated hydrocarbon having the indicated number of carbon atoms and at least one triple bond. Examples of a (C₂-C₈)alkynyl group include, but are not limited to, acetylene, propyne, 1-butyne, 2-butyne, 1-pentyne, 2-pentyne, 1-hexyne, 2-hexyne, 3-hexyne, 1-heptyne, 2-heptyne, 3-heptyne, 1-octyne, 2-octyne, 3-octyne and 4-octyne. An alkynyl group can be unsubstituted or optionally substituted with one or more substituents as described herein below.

The term “alkoxy” refers to an —O-alkyl group having the indicated number of carbon atoms. For example, a (C₁-C₆)alkoxy group includes —O-methyl, —O-ethyl, —O-propyl, —O-isopropyl, —O-butyl, —O-sec-butyl, —O-tert-butyl, —O-pentyl, —O-isopentyl, —O-neopentyl, —O-hexyl, —O-isohexyl, and —O-neohexyl.

The term “aryl” refers to a 6- to 18-membered bicyclic, tricyclic, or polycyclic aromatic hydrocarbon ring system. Examples of an aryl group include naphthyl, pyrenyl, and anthracyl. An aryl group can be unsubstituted or optionally substituted with one or more substituents as described herein below.

The terms “heterocycle” and “heterocycloalkyl” refer to bicyclic, tricyclic, or polycyclic 8- to 14-membered ring systems, which are either unsaturated or aromatic and which contains from 1 to 4 heteroatoms, independently selected from nitrogen, oxygen and sulfur, wherein the nitrogen and sulfur heteroatoms are optionally oxidized and the nitrogen heteroatom optionally quaternized, including bicyclic, and tricyclic ring systems. The bicyclic or tricyclic ring systems may be spiro-fused. The bicyclic and tricyclic ring systems may encompass a heterocycle or heteroaryl fused to a benzene ring. The heterocycle may be attached via any heteroatom or carbon atom. Heterocycles include heteroaryls as defined above. Representative examples of heterocycles include, but are not limited to, benzoxazolyl, benzisoxazolyl, benzthiazolyl, benzimidazolyl, isoindolyl, indazolyl, benzodiazolyl, benzotriazolyl, benzoxazolyl, benzisoxazolyl, purinyl, indolyl, isoquinolinyl, quinolinyl and quinazolinyl. A heterocycle group can be unsubstituted or optionally substituted with one or more substituents as described herein below.

The term “halogen” and “halo” refers to —F, —Cl, —Br or —I.

The term “haloalkyl,” refers to a C₁-C₆ alkyl group wherein from one or more of the C₁-C₆ alkyl group's hydrogen atom is replaced with a halogen atom, which can be the same or different. Examples of haloalkyl groups include, but are not limited to, trifluoromethyl, 2,2,2-trifluoroethyl, 4-chlorobutyl, 3-bromopropyl, pentachloroethyl, and 1,1,1-trifluoro-2-bromo-2-chloroethyl.

The term “heteroaryl” denotes a polycyclic aromatic heterocyclic ring system ring of 5 to 18 members, having at least one heteroatom selected from nitrogen, oxygen and sulfur, and containing at least 1 carbon atom, including bicyclic, and tricyclic ring systems. Examples of heteroaryls are benzofuranyl, benzothiophenyl, quinolinyl, indolyl, benzoxazolyl, benzimidazolyl, benzothiazolyl, pyrimidinyl, cinnolinyl, phthalazinyl, quinazolinyl, pyrimidyl, chromenonyl, quinoxalinyl. A heteroaryl group can be unsubstituted or optionally substituted with one or more substituents as described herein below.

The term “heteroatom” is meant to include oxygen (O), nitrogen (N), and sulfur (S).

The term “hydroxyalkyl,” refers to an alkyl group having the indicated number of carbon atoms wherein one or more of the alkyl group's hydrogen atoms is replaced with an —OH group. Examples of hydroxyalkyl groups include, but are not limited to, —CH₂OH, —CH₂CH₂OH, —CH₂CH₂CH₂OH, —CH₂CH₂CH₂CH₂OH, —CH₂CH₂CH₂CH₂CH₂OH, —CH₂CH₂CH₂CH₂CH₂CH₂OH, and branched versions thereof.

Substituents for the groups referred to as alkyl, heteroalkyl, alkylene, alkenyl, and alkynyl can be a variety of groups selected from: —OR′, ═O, ═NR′, ═N—OR′, —NR′R″, —SR′, —halo, —SiR′R″R′″, —OC(O)R′, —C(O)R′, —CO₂R′, —CONR′R″, —OC(O)NR′R″, —NR″C(O)R′, —NR′″C(O)NR′R″, —NR′″SO₂NR′R″, —NR″CO₂R′, —NHC(NH₂)═NH, —NR′C(NH₂)═NH, —NHC(NH₂)═NR′, —S(O)R′, —SO₂R′, —SO₂NR′R″, —NR″SO₂R′, —CN and —NO₂, in a number ranging from zero to three, with those groups having zero, one or two substituents being exemplary. R′, R″ and R′″ each independently refer to hydrogen, unsubstituted (C₁-C₈)alkyl, unsubstituted hetero(C₁-C₈)alkyl, unsubstituted aryl and aryl substituted with one to three substituents selected from -halo, unsubstituted alkyl, unsubstituted alkoxy, unsubstituted thioalkoxy and unsubstituted aryl(C₁-C₄)alkyl. When R′ and R″ are attached to the same nitrogen atom, they can be combined with the nitrogen atom to form a 5-, 6- or 7-membered ring. For example, —NR′R″ is meant to include 1-pyrrolidinyl and 4-morpholinyl. An alkyl or heteroalkyl group will have from zero to three substituents, with those groups having two or fewer substituents being exemplary in the present invention. In some embodiments, an alkyl or heteroalkyl radical will be unsubstituted or monosubstituted. An alkyl or heteroalkyl radical can be unsubstituted. From the above discussion of substituents, one of skill in the art will understand that the term “alkyl” is meant to include groups such as trihaloalkyl (e.g., —CF₃ and —CH₂CF₃).

Exemplary substituents for the alkyl and heteroalkyl radicals are selected from: —OR′, ═O, —NR′R″, —SR′, -halo, —SiR′R″R′″, —OC(O)R′, —C(O)R′, —CO₂R′, —C(O)NR′R″, —OC(O)NR′R″, —NR″C(O)R′, —NR″CO₂R′, —NR′″SO₂NR′R″, —S(O)R′, —SO₂R′, —SO₂NR′R″, —NR″SO₂R′, —CN and —NO₂, where R′, R″ and R′″ are as defined above. Typically, substituents are selected from: —OR′, ═O, —NR′R″, -halo, —OC(O)R′, —CO₂R′, —C(O)NR′R″, —OC(O)NR′R″, —NR″C(O)R′, —NR″CO₂R′, —NR′″SO₂NR′R″, —SO₂R′, —SO₂NR′R″, —NR″SO₂R′—CN and —NO₂.

Similarly, substituents for the aryl and heteroaryl groups are varied and selected from: -halo, —OR′, —OC(O)R′, —NR′R″, —SR′, —R′, —CN, —NO₂, —CO₂R′, —C(O)NR′R″, —C(O)R′, —OC(O)NR′R″, —NR″C(O)R′, —NR″CO₂R′, —NR′″C(O)NR′R″, —NR′″SO₂NR′R″, —NHC(NH₂)═NH, —NR′C(NH₂)═NH, —NH—C(NH₂)═NR′, —S(O)R′, —SO₂R′, —SO₂NR′R″, —NR″SO₂R′, —N₃, —CH(Ph)₂, perfluoroalkoxy and perfluoro(C₁-C₄)alkyl, in a number ranging from zero to the total number of open valences on the aromatic ring system; and where R′, R″ and R′″ are independently selected from hydrogen, unsubstituted (C₁-C₈)alkyl, unsubstituted hetero(C₁-C₈)alkyl, unsubstituted aryl, unsubstituted heteroaryl, unsubstituted aryl(C₁-C₄)alkyl and unsubstituted aryloxy(C₁-C₄)alkyl. Typically, an aryl or heteroaryl group will have from zero to three substituents, with those groups having two or fewer substituents being exemplary in the present invention. In one embodiment of the invention, an aryl or heteroaryl group will be unsubstituted or monosubstituted. In another embodiment, an aryl or heteroaryl group will be unsubstituted.

Exemplary substituents for aryl and heteroaryl groups are selected from: -halo, —OR′, —OC(O)R′, —NR′R″, —SR′, —R′, —CN, —NO₂, —CO₂R′, —CONR′R″, —C(O)R′, —OC(O)NR′R″, —NR″C(O)R′, —S(O)R′, —SO₂R′, —SO₂NR′R″, —NR″SO₂R′, —N₃, —CH(Ph)₂, perfluoroalkoxy and perfluoro(C₁-C₄)alkyl, where R′ and R″ are as defined above. Typically, substituents are selected from: -halo, —OR′, —OC(O)R′, —NR′R″, —R′, —CN, —NO₂, —CO₂R′, —CONR′R″, —NR″C(O)R′, —SO₂R′, —SO₂NR′R″, —NR″SO₂R′, perfluoroalkoxy and perfluoro(C₁-C₄)alkyl.

Two of the substituents on adjacent atoms of the aryl or heteroaryl ring may optionally be replaced with a substituent of the formula -T-C(O)—(CH₂)_{q}—U—, wherein T and U are independently —NH—, —O—, —CH₂— or a single bond, and q is an integer of from 0 to 2. Alternatively, two of the substituents on adjacent atoms of the aryl or heteroaryl ring may optionally be replaced with a substituent of the formula -A-(CH₂)ᵣ—B—, wherein A and B are independently —CH₂—, —O—, —NH—, —S—, —S(O)—, —S(O)₂—, —S(O)₂NR′— or a single bond, and r is an integer of from 1 to 3. One of the single bonds of the new ring so formed may optionally be replaced with a double bond. Alternatively, two of the substituents on adjacent atoms of the aryl or heteroaryl ring may optionally be replaced with a substituent of the formula —(CH₂)ₛ—X—(CH₂)ₜ—, where s and t are independently integers of from 0 to 3, and X is —O—, —NR′—, —S—, —S(O)—, —S(O)₂—, or —S(O)₂NR′—. The substituent R′ in —NR′— and —S(O)₂NR′— is selected from hydrogen or unsubstituted (C₁-C₆)alkyl.

The substituent —CO₂H, may be replaced with bioisosteric replacements such as:

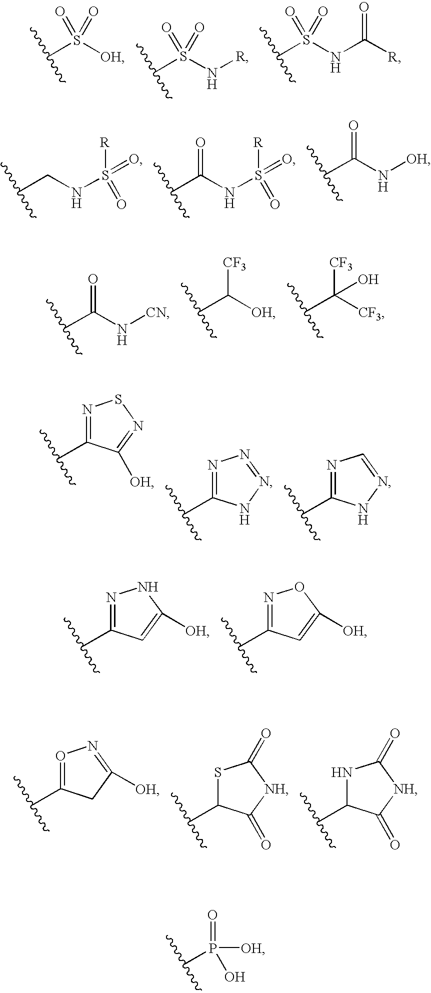

and the like. See, e.g., THE PRACTICE OF MEDICINAL CHEMISTRY (Academic Press: New York, 1996), at page 203.

The compound of the invention can also exist in various isomeric forms, including configurational, geometric, and conformational isomers, as well as existing in various tautomeric forms, particularly those that differ in the point of attachment of a hydrogen atom. The term “isomer” is intended to encompass all isomeric forms of a compound of formula I, including tautomeric forms of the compound.

Certain compounds as herein defined may have asymmetric centers and therefore exist in different enantiomeric and diastereomeric forms. A compound of the invention can be in the form of an optical isomer or a diastereomer. Accordingly, the invention encompasses compounds of formula I and their uses as described herein in the form of their optical isomers, diasterisomers and mixtures thereof, including a racemic mixture. Optical isomers of the compounds of the invention can be obtained by known techniques such as asymmetric synthesis, chiral chromatography, simulated moving bed technology or via chemical separation of stereoisomers through the employment of optically active resolving agents.

Unless otherwise indicated, the term “stereoisomer” or means one stereoisomer of a compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound.

If there is a discrepancy between a depicted structure and a name given that structure, then the depicted structure controls. In addition, if the stereochemistry of a structure or a portion of a structure is not indicated with, for example, bold or dashed lines, the structure or portion of the structure is to be interpreted as encompassing all stereoisomers of it.

In this description, a “pharmaceutically acceptable salt” is a pharmaceutically acceptable, organic or inorganic acid or base salt of a compound of the invention. Representative pharmaceutically acceptable salts include, e.g., alkali metal salts, alkali earth salts, ammonium salts, water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts. A pharmaceutically acceptable salt can have more than one charged atom in its structure. In this instance the pharmaceutically acceptable salt can have multiple counterions. Thus, a pharmaceutically acceptable salt can have one or more charged atoms and/or one or more counterions.

The term “prodrug” denotes a derivative of a compound that can hydrolyze, oxidize, or otherwise react under biological conditions, in vitro or in vivo, to provide an active compound, particularly a compound of the invention. Examples of prodrugs include, but are not limited to, derivatives and metabolites of a compound of formula I that include biohydrolyzable groups such as biohydrolyzable amides, biohydrolyzable esters, biohydrolyzable carbamates, biohydrolyzable carbonates, biohydrolyzable ureides, and biohydrolyzable phosphate analogues (e.g., monophosphate, diphosphate or triphosphate). For instance, prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by BURGER'S MEDICINAL CHEMISTRY AND DRUG DISCOVERY 6^{th} ed. (Wiley, 2001) and DESIGN AND APPLICATION OF PRODRUGS (Harwood Academic Publishers Gmbh, 1985).

The terms “treat”, “treating” and “treatment” refer to the amelioration or eradication of a disease or symptoms associated with a disease. In certain embodiments, such terms refer to minimizing the spread or worsening of the disease resulting from the administration of one or more prophylactic or therapeutic agents to a patient with such a disease.

The terms “prevent,” “preventing” and “prevention” refer to the prevention of the onset, recurrence, or spread of the disease in a patient resulting from the administration of a prophylactic or therapeutic agent.

The term “effective amount” refers to an amount of a compound of the invention or other active ingredient sufficient to provide a therapeutic or prophylactic benefit in the treatment or prevention of a disease or to delay or minimize symptoms associated with a disease. Further, a therapeutically effective amount with respect to a compound of the invention means that amount of therapeutic agent alone, or in combination with other therapies, that provides a therapeutic benefit in the treatment or prevention of a disease. Used in connection with a compound of the invention, the term can encompass an amount that improves overall therapy, reduces or avoids symptoms or causes of disease, or enhances the therapeutic efficacy of or synergies with another therapeutic agent.

The terms “modulate”, “modulation” and the like refer to the ability of a compound to increase or decrease the function, or activity of, for example, DHODH. “Modulation”, in its various forms, is intended to encompass inhibition, antagonism, partial antagonism, activation, agonism and/or partial agonism of the activity associated with DHODH. DHODH inhibitors are compounds that, e.g., bind to, partially or totally block stimulation, decrease, prevent, delay activation, inactivate, desensitize, or down regulate signal transduction. DHODH activators are compounds that, e.g., bind to, stimulate, increase, open, activate, facilitate, enhance activation, sensitize or up regulate signal transduction. The ability of a compound to modulate DHODH can be demonstrated in an enzymatic assay or a cell-based assay.

A “patient” includes an animal (e.g., cow, horse, sheep, pig, chicken, turkey, quail, cat, dog, mouse, rat, rabbit or guinea pig), in one embodiment a mammal such as a non-primate and a primate (e.g., monkey and human), and in another embodiment a human. In one embodiment, a patient is a human. In specific embodiments, the patient is a human infant, child, adolescent or adult.

### Compounds of the Pharmaceutical Compositions and Methods of the Invention

The present invention provides pharmaceutical compositions comprising a compound of the formula

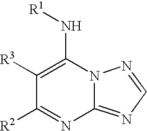

or a pharmaceutically acceptable salt, solvate, tautomer, stereoisomer, or prodrug, wherein all the variables are defined as above.

In one embodiment, the compound is one according to Formula (I)

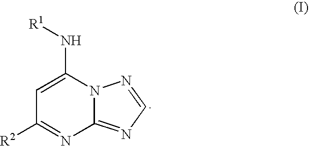

In one embodiment, R¹ is aryl. In another embodiment, R¹ is heteroaryl. In still another embodiment, R¹ is (C₈-C₁₄) heterocycloalkyl.

In one embodiment, R² is (C₁-C₃)alkyl, in particular, methyl.

In another embodiment, R² is (C₁-C₃)haloalkyl, in particular, trifluoromethyl.

In still another embodiment, R¹ is selected from aryl, heteroaryl, and (C₈-C₁₄)heterocycloalkyl and R² is (C₁-C₃)alkyl.

In yet another embodiment, each of R² and R³ is (C₁-C₃)alkyl. For instance, each of R² and R³ can be methyl.

In one embodiment, the pharmaceutical composition of the invention further comprises an additional therapeutic agent. For example, the additional therapeutic agent may be a pyrimidine biosynthesis inhibitor.

Examples of compounds of the invention are provided in Table 1 below.

TABLE 1

| | IC₅₀ pf DHODH | | IC₅₀ hDHODH | | EC₅₀ P. falciparum | |
|---|---|---|---|---|---|---|
| Structure | (nM) | | (nM) | | 3D7 in vitro (nM) | |
| 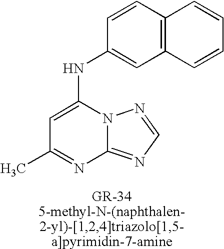 | 40 | nM | >600,000 | nM | 50 | nM |
| 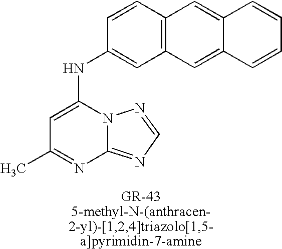 | 23 | nM | >200,000 | nM | 120 | nM |
| 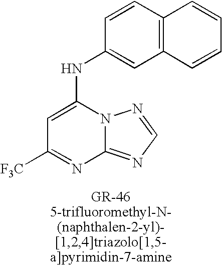 | 126 | nM | >200,000 | nM | | |
| 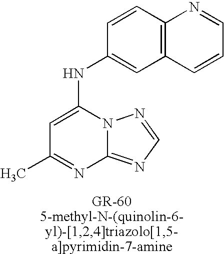 | 1,300 | nM | >200,000 | nM | | |
| 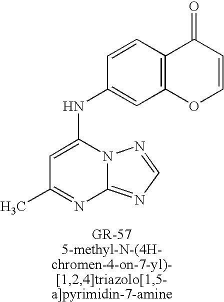 | 1,900 | nM | >200,000 | nM | | |
| 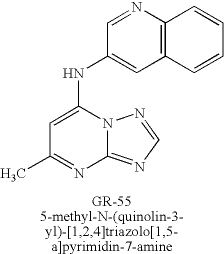 | 2,000 | nM | >200,000 | nM | | |
| 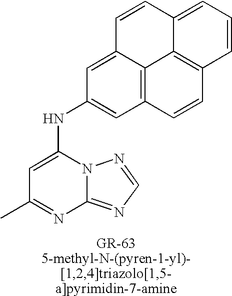 | 2,100 | nM | >50,000 | nM | | |
| 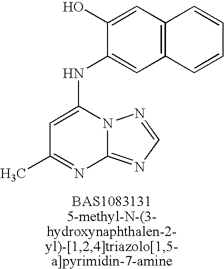 | 2,400 | nM | | | 600 | nM |
| 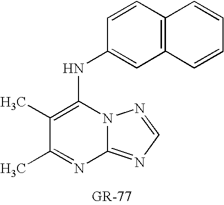 | 130 | nM | | | | |
| 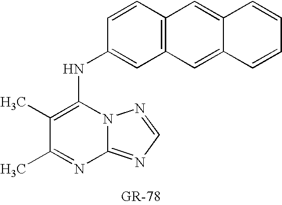 | 250 | nM | | | | |
| 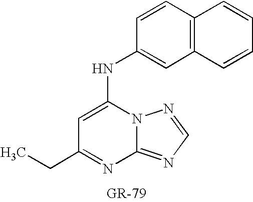 | 280 | nM | | | | |
| 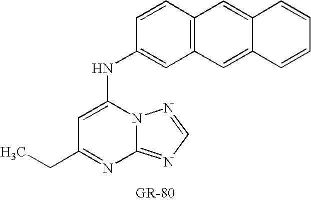 | 240 | nM | | | | |

In one embodiment, the pharmaceutical composition of the invention comprises 5-methyl-N-(naphthalen-2-yl)-[1,2,4]triazolo[1,5-a]pyrimidin-7-amine or a pharmaceutically acceptable salt, solvate, stereoisomer, tautomer, or prodrug thereof.

In one embodiment, the invention provides methods of inhibiting dihydroororate dehydrogenase in a parasite, comprising contacting said parasite with a pharmaceutical composition comprising a compound of the invention. In one embodiment, the parasite is a member of the Plasmodium genus. In another embodiment, the parasite is Plasmodium falciparum.

In another embodiment, the invention provides methods of treating or preventing malaria, inhibiting dihydroororate dehydrogenase in a parasite, such as Plasmodium falciparum, in vitro or in vivo, or killing a Plasmodium falciparum parasite, wherein the pharmaceutical composition comprises a compound selected from the group consisting of
5-methyl-N-(naphthalen-2-yl)-[1,2,4]triazolo[1,5-a]pyrimidin-7-amine;
5-methyl-N-(anthracen-2-yl)-[1,2,4]triazolo[1,5-a]pyrimidin-7-amine;
5-trifluoromethyl-N-(naphthalen-2-yl)-[1,2,4]triazolo[1,5-a]pyrimidin-7-amine;
5-methyl-N-(quinolin-6-yl)-[1,2,4]triazolo[1,5-a]pyrimidin-7-amine;
5-methyl-N-(4H-chromen-4-on-7-yl)-[1,2,4]triazolo[1,5-a]pyrimidin-7-amine;
5-methyl-N-(quinolin-3-yl)-[1,2,4]triazolo[1,5-a]pyrimidin-7-amine;
5-methyl-N-(pyren-1-yl)-[1,2,4]triazolo[1,5-a]pyrimidin-7-amine;
5-methyl-N-(3-hydroxynaphthalen-2-yl)-[1,2,4]triazolo[1,5-a]pyrimidin-7-amine, and
5,6-dimethyl-N-(naphthalen-2-yl)-[1,2,4]triazolo[1,5-a]pyrimidin-7-amine;
N-(anthracen-2-yl)-5,6-dimethyl-[1,2,4]triazolo[1,5-a]pyrimidin-7-amine;
5-ethyl-N-(naphthalen-2-yl)-[1,2,4]triazolo[1,5-a]pyrimidin-7-amine; and
N-(anthracen-2-yl)-5-ethyl-[1,2,4]triazolo[1,5-a]pyrimidin-7-amine;
or a pharmaceutically acceptable salt, solvate, stereoisomer, tautomer, or prodrug thereof.
   Pharmaceutical Compositions and Dosage

Pharmaceutical compositions and single unit dosage forms comprising a compound of the invention, or a pharmaceutically acceptable stereoisomer, prodrug, salt, solvate, hydrate, tautomer, or clathrate thereof, are also encompassed by the invention.

In accordance with this invention, the aforementioned compounds of the invention or their pharmaceutically acceptable salts are useful in pharmaceutically acceptable compositions. These pharmaceutical compositions of the invention contain said compound of the invention or its pharmaceutically acceptable salts, in association with a compatible pharmaceutically acceptable carrier material. Any conventional carrier material can be utilized. The carrier material can be an organic or inorganic inert carrier material, for example one that is suitable for oral administration. Suitable carriers include water, gelatin, gum arabic, lactose, starch, magnesium stearate, talc, vegetable oils, polyalkylene-glycols, petroleum jelly and the like. Furthermore, the pharmaceutical preparations may also contain other pharmaceutically active agents. Additional additives such as flavoring agents, preservatives, stabilizers, emulsifying agents, buffers and the like may be added in accordance with accepted practices of pharmaceutical compounding.

The pharmaceutical preparations can be made up in any conventional form including a solid form for oral administration such as tablets, capsules, pills, powders, granules, and the like. The pharmaceutical preparations may be sterilized and/or may contain adjuvants such as preservatives, stabilizers, wetting agents, emulsifiers, salts for varying the osmotic pressure and/or buffers.

The compounds of the invention can also be administered to a patient in accordance with the invention by topical (including transdermal, buccal or sublingual), or parenteral (including intraperitoneal, subcutaneous, intravenous, intradermal or intramuscular injection) routes. In one embodiment, the compounds of formula I are administered orally. An oral dosage form comprises tablets, capsules of hard or soft gelatin methylcellulose or of another suitable material easily dissolved in the digestive tract. The oral dosages contemplated in accordance with the present invention will vary in accordance with the needs of the individual patient as determined by the prescribing physician. For example, a daily dosage of from about 1 mg to about 50 mg per kg of body weight, such as from about 5 mg to about 25 mg per kg of body weight of the patient may be utilized.

It is within the purview of the present invention to incorporate the therapeutically active substance enumerated herein in any desired mount for enteral administration within the oral unit dosage form. For enteral or oral administration, particularly suitable are tablets, dragees or capsules having talc and/or carbohydrate carrier binder or the like, the carrier could be lactose and/or corn starch and/or potato starch. A syrup, elixir or the like can be used where a sweetened vehicle is employed. Sustained release compositions can be formulated including those where the active component is protected with differentially degradable coatings, e.g., by microencapsulation, multiple coatings, etc. For example, preparations containing the active substance of the present invention can be formulated in such a manner that each dose forms contains from about 50 mg to about 1000 mg, or about 250 mg, with suitable therapeutically inert fillers and dilutents.

For parenteral application, particularly suitable are solutions, preferably oily or aqueous solutions as well as suspensions, emulsions, or implants, including suppositories. Therapeutic compounds will be formulated in sterile form in multiple or single dose formats such as being dispersed in a fluid carrier such as sterile physiological saline or 5% saline dextrose solutions commonly used with injectables.

For topical applications, the compound(s) of the invention can be suitably admixed in a pharmacologically inert topical carrier such as a gel, an ointment, a lotion or a cream. Such topical carriers include water, glycerol, alcohol, propylene glycol, fatty alcohols, triglycerides, fatty acid esters, or mineral oils. Other possible topical carriers are liquid petrolatum, isopropylpalmitate, polyethylene glycol, ethanol 95%, polyoxyethylene monolauriate 5% in water, sodium lauryl sulfate 5% in water, and the like. In addition, materials such as anti-oxidants, humectants, viscosity stabilizers and the like also may be added if desired.

The actual preferred amounts of active compounds used in a given therapy will vary according to the specific compound being utilized, the particular compositions formulated, the mode of application, the particular site of administration, etc. Optimal administration rates for a given protocol of administration can be readily ascertained by those skilled in the art using conventional dosage determination tests conducted with regard to the foregoing guidelines. The dosage for treatment typically depends on the route of administration, the age, weight and degree of malarial infection of the patient.

In general, compounds of the invention for treatment can be administered to a subject in dosages used in prior malaria therapies. See, for example, the Physicians' Desk Reference. For example, a suitable effective dose of one or more compounds of the invention will be in the range of from 0.01 to 100 milligrams per kilogram of body weight of recipierit per day, preferably in the range of from 1 to 50 milligrams per kilogram body weight of recipient per day, more preferably in the range of 5 to 25 milligrams per kilogram body weight of recipient per day. The desired dose is suitably administered once daily, or several sub-doses, e.g. 2 to 5 sub-doses, are administered at appropriate intervals through the day, or other appropriate schedule.

Therapeutic Uses of the Pharmaceutical Compositions of Formula I

In one aspect, the invention provides methods of treating or preventing a condition or disorder associated with inhibition of Plasmodium dihydroorotate dehydrogenase by administering to a patient having such a condition or disorder a therapeutically effective amount of a composition of the invention. In one group of embodiments, the conditions or disorders, including diseases of humans, can be treated with inhibitors of Plasmodium DHODH, such as P. falciparum dihydroorotate dehydrogenase (pfDHODH).

Treatment of Malaria

Malaria can be treated or prevented by administration of a therapeutically effective amount of a compound of formula I or composition comprising a compound of formula I.

Additional Therapeutic Agents

In one embodiment, the present methods for treating or preventing malaria further comprise the administration of a therapeutically effective amount of another therapeutic agent useful for inhibiting pyrimidine synthesis. In this embodiment, the time in which the therapeutic effect of the other therapeutic agent is exerted overlaps with the time in which the therapeutic effect of the compound of formula I is exerted.

The compositions of the invention can be combined or used in combination with other agents useful in the treatment, prevention, suppression or amelioration of malaria.

Such other agents, or drugs, may be administered, by a route and in an amount commonly used therefor, simultaneously or sequentially with a composition comprising a compound of the invention. In one embodiment, a pharmaceutical composition contains such other drugs in addition to the compound of the invention when a compound of the invention is used contemporaneously with one or more other drugs. Accordingly, the pharmaceutical compositions of the invention include those that also contain one or more other active ingredients or therapeutic agents, in addition to a compound of the invention.

In one embodiment, for the treatment or prevention of malaria, a compound of the invention can be administered with another therapeutic agent. The additional therapeutic agent may treat malaria directly, headache, malaise, anemia, splenomegaly, and/or fever. Examples of additional therapeutic agents include proguanil, chlorproguanil, trimethoprim, chloroquine, mefloquine, lumefantrine, atovaquone, pyrimethamine-sulfadoxine, pyrimethamine-dapsone, halofantrine, quinine, quinidine, amodiaquine, amopyroquine, sulphonamides, artemisinin, arteflene, artemether, artesunate, primaquine, pyronaridine, and combinations thereof.

The present invention is not to be limited in scope by the specific embodiments disclosed in the examples which are intended as illustrations of a few embodiments of the invention and any embodiments that are functionally equivalent are within the scope of this invention. Indeed, various modifications of the invention in addition to those shown and described herein will become apparent to those skilled in the art and are intended to fall within the scope of the appended claims. To this end, it should be noted that one or more hydrogen atoms or methyl groups may be omitted from the drawn structures consistent with accepted shorthand notation of such organic compounds, and that one skilled in the art of organic chemistry would readily appreciate their presence.

### EXAMPLE 1

### Synthesis of 5-methyl-N-(naphthalen-2-yl)-[1,2,4]triazolo[1,5-a]pyrimidin-7-amine

The following synthesis, yielding a compound designated “GR-34” (see Table 1), illustrates how all compounds as herein described are produced, in accordance with the present invention.

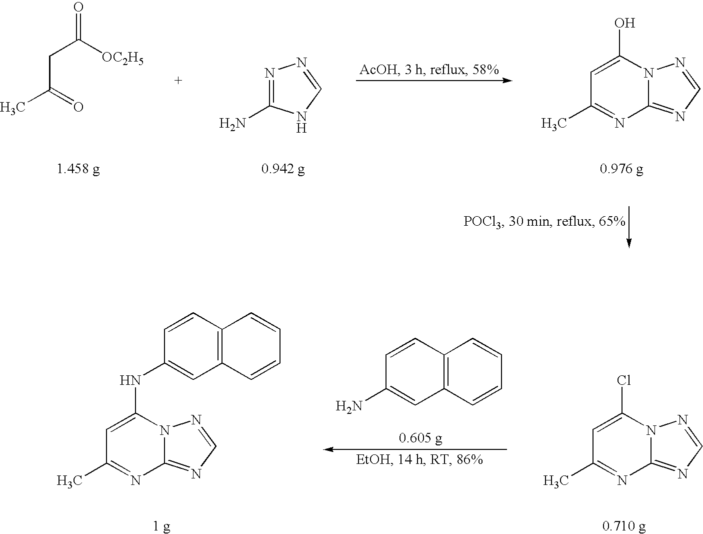

5-Methyl-[1,2,4]triazolo[1,5-a]pyrimidin-7-ol: A mixture of 0.942 g (11.20 mmol) 1,2,4-triazole and 1.458 g (11.20 mmol) of ethyl acetoacetate was heated under reflux in 10 mL of acetic acid for 3 hours. The reaction then was cooled to room temperature. A white solid was isolated by filtration, washed with water, and dried under vacuum to give the product with 58% (0.976 g) yield.

7-Chloro-5-methyl-[1,2,4]triazolo[1,5-a]pyrimidine: To 0.976 g (6.50 mmol) 5-Methyl-[1,2,4]triazolo[1,5-a]-pyrimidin-7-ol, in a round bottom flask, was added 1.82 mL (19.50 mmol) of phosphorus oxychloride, and the mixture was heated under reflux for 30 minutes, during which time the solid dissolved and hydrogen chloride evolved. The excess phosphorus oxychloride was removed by distillation at reduced pressure on a steam-bath, and the residue triturated with ice water. The product was extracted from the aqueous mixture with methylene chloride. The resulting solution was evaporated and purified by column chromatography to give the product with 65% (0.710 g) yield.

(5-Methyl-11,2,4]triazolo[1,5-a]pyrimidin-7-yl)-naphthalen-2-yl-amine: Naphthyl amine (0.605 g, 4.22 mmol) was added to the stirred solution of 7-chloro-5-methyl-[1,2,4]triazolo[1,5-a]pyrimidine (0.710 g, 4.22 mmol) in 10 mL of absolute ethanol. The stirring was continued for 14 hours at room temperature. The expected product was crystallized from ethanol solvent and filtered. The crude product was purified by column chromatography to give product in 86% yield (1 g).

[image]

[image]

[image]

### EXAMPLE 2

### Procedures Useful for the Biological Evaluation of the Compounds of Formula I

In addition to the extensive literature disclosing the role of DHOHD in malaria, described here are assays useful for testing the compounds of Formula I of the present invention.

Assays

Measurement of enzyme inhibition. For studying inhibition of Plasmodium or human DHODH enzyme, two assays that are in routine use are described, for example, in Baldwin, et al. (2002) J Biol Chem., 277, 41827-41834, and Baldwin, et al. (2005) J. Biol. Chem., 280. 21847-21853. Briefly, these assays are as follows: 1) A calorimetric assay thay that monitors 2,6-dichloroindophenol (DCIP) reduction at 600 nm (e=18.8 mM⁻¹ cm⁻¹) is performed as either a continuous assay where absorbance is recorded over time. The assay solution containing 100 mM HEPES, pH 8.0, 150 mM NaCl, 10% glycerol, 0.05% Triton X-100,20 mM CoQ₀ (coenzyme Q_{D}), 200 mM L-dihydroorotate, and 120 mM DCIP. Reactions are initiated by addition of enzyme to a final concentration of 5-50 nM and the temperature is maintained at 25° C with a circulating water bath. 2) A direct assay that follows the production of orotic acid by measuring the absorbance change at 287 nm (ε=4.3 mM⁻¹ cm⁻¹); DCIP is not included, but all other components of the assay are as described in method one. Inhibitor stocks are prepared in DMSO, and the inhibitors, such as GR-34, are added to these assays over a concentration range that encompasses their IC₅₀ value and, the IC₅₀'s are determined from a plot of reaction rate in the presence of inhibitor/reaction rate in the absence of inhibitor (vi/vo) versus inhibitor concentration (Eq. 1).

$${v}_{i} = \frac{{v}_{o}}{1 + \frac{[ I ]}{{IC}_{50}}} \qquad Equation 1$$

In vitro evaluation of compound efficacy on the human malaria parasite, P. falciparum. To study inhibition of cell proliferation, ³H-hypoxanthine uptake is measured in drug-treated, P. falciparum-infected erythrocytes grown in culture, pursuant to the methodology of Desjardins, et al. (1979) Antimicrobial Agents and Chemotherapy 16, 710-718, and Zhang and Rathod (2002) Science 296, 545-547.

In vivo Evaluation of Compound Efficacy

(A) The standard P. berghei mouse model for infection will be utilized to evaluate the efficacy of candidate compounds, according to the invention, against parasites in vivo. See review of Fidock, et al. (2004) Nature Rev. Drug Discovery 3, 509-20. Compounds will be dosed either orally or IP, with the exact regimens (e.g. frequency of dosing, drug concentrations at dosing) determined based on the pharmocokinetic profiles of the individual analogs.

(B) In the event that the mouse model does not provide a positive indication for a given candidate compound, the preferred course of action will be to determine whether P. bergehi grown in vitro, in short term culture, is sensitive to the candidate. If sensitivity of P. berghei proves to be the key issue, then a genetically altered P. berghei strain, containing the P. falciparum DHODH enzyme, will be generated for the in vivo testing. See Braks, et al. (2006) Nucleic Acids Res. 34, e39, and Janse, et al. (2006) Mol. Biochem. Parasitol. 145, 60-70. Alternatively, the humanized malarial mouse model will be used for the testing, in accordance with Morosan, et al. (2006) J. Infect. Dis. 193, 996-1004.

A composition of the present invention, comprising a formula I compound, typically assays with pfDHODH enzyme activity in a range from about 2500 nm to <25 nm, and displays an in vitro activity, against cultured P. falciparum malaria parasite, ranging from about 2000 nM to about <10 nM. The compounds of formula I exhibit selectivity of about 10² to about 10⁵ times greater for the pfDHODH enzyme over the hDHODH enzyme.

What is claimed is:

1. A pharmaceutical composition comprising
(a) a compound of the formula 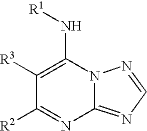
or pharmaceutically acceptable salts, solvates, stereoisomers, tautomers, or prodrugs thereof, wherein
R¹ is selected from the group consisting of (C₈-C₁₄) heterocycloalkyl, aryl, and heteroaryl, wherein the heterocycloalkyl, aryl or heteroaryl has two or more rings; and
R² and R³ are independently selected from the group consisting of halogen, (C₁-C₈)alkyl, (C₂-C₈)alkenyl, (C₂-C₈)alkynyl, (C₁-C₈)alkoxy, and (C₁-C₈)haloalkyl;
wherein any heterocycloalkyl, aryl or heteroaryl is optionally substituted with one or more members selected from the group consisting of halogen, —CN, —NO₂, hydroxyl, (C₁-C₈)alkyl, (C₂-C₈)alkenyl, (C₂-C₈)alkynyl, (C₁-C₄)alkoxy, (C₁-C₄)haloalkyl, and (C₂-C₄)hydroxyalkyl; and
(b) a pharmaceutically acceptable carrier.

2. A pharmaceutical composition of claim 1, comprising
(a) a compound of formula (I) 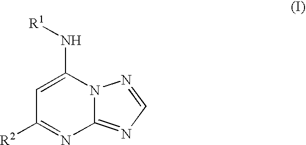
or pharmaceutically acceptable salts, solvates, stereoisomers, tautomers, or prodrugs thereof, wherein
R¹ is selected from the group consisting of (C₈-C₁₄) heterocycloalkyl, aryl, and heteroaryl, wherein the heterocycloalkyl, aryl or heteroaryl has two or more rings; and
R² is selected from the group consisting of halogen, (C₁-C₈)alkyl, (C₂-C₈)alkenyl, (C₂-C₈)alkynyl, (C₁-C₈)alkoxy, and (C₁-C₈)haloalkyl;
wherein any heterocycloalkyl, aryl or heteroaryl is optionally substituted with one or more members selected from the group consisting of halogen, —CN, —NO₂, hydroxyl, (C₁-C₈)alkyl, (C₂-C₈)alkenyl, (C₂-C₈)alkynyl, (C₁-C₄)alkoxy, (C₁-C₄)haloalkyl, and (C₂-C₄)hydroxyalkyl; and
(b) a pharmaceutically acceptable carrier.

3. The pharmaceutical composition of claim 2, wherein R¹ is aryl.

4. The pharmaceutical composition of claim 2, wherein R¹ is heteroaryl.

5. The pharmaceutical composition of claim 2, wherein R¹ is (C₈-C₁₄)heterocycloalkyl.

6. The pharmaceutical composition of claim 2, wherein R² is (C₁-C₃)alkyl.

7. The pharmaceutical composition of claim 6, wherein R² is methyl.

8. The pharmaceutical composition of claim 1, wherein each of R² and R³ is (C₁-C₃)alkyl.

9. The pharmaceutical composition of claim 8, wherein each of R² and R³ is methyl.

10. The pharmaceutical composition of claim 1, further comprising an additional therapeutic agent.

11. The pharmaceutical composition of claim 10, wherein the additional therapeutic agent is a pyrimidine biosynthesis inhibitor.

12. A method for the treatment of malaria, comprising administering to a patient in need thereof a therapeutically effective amount of a pharmaceutical composition of claim 1.

13. A method of inhibiting dihydroororate dehydrogenase in a parasite, comprising contacting said parasite with a pharmaceutical composition of claim 1.

14. The method of claim 13, wherein the parasite is a member of the Plasmodium genus.

15. The method of claim 14, wherein the parasite is Plasmodium falciparum.

16. A method of inhibiting dihydroororate dehydrogenase of a malaria parasite in a host mammal, comprising administering to the host mammal an effective amount of a pharmaceutical composition of claim 1, whereby mammalian dihydroororate dehydrogenase is not inhibited.

17. A method of killing a Plasmodium falciparum parasite comprising contacting said parasite with an effective amount of a pharmaceutical composition of claim 1.

18. A method of killing Plasmodium falciparum parasites in a host mammal comprising administering to the host mammal in need thereof a therapeutically effective amount of a pharmaceutical composition of claim 1.

19. A pharmaceutical composition comprising
(a) a compound selected from the group consisting of:
5-methyl-N-(naphthalen-2-yl)-[1,2,4]triazolo[1,5-a]pyrimidin-7-amine;
5-methyl-N-(anthracen-2-yl)-[1,2,4]triazolo[1,5-a]pyrimidin-7-amine;
5-trifluoromethyl-N-(naphthalen-2-yl)-[1,2,4]triazolo[1,5-a]pyrimidin-7-amine;
5-methyl-N-(quinolin-6-yl)-[1,2,4]triazolo[1,5-a]pyrimidin-7-amine;
5-methyl-N-(4H-chromen-4-on-7-yl)-[1,2,4]triazolo[1,5-a]pyrimidin-7-amine;
5-methyl-N-(quinolin-3-yl)-[1,2,4]triazolo[1,5-a]pyrimidin-7-amine;
5-methyl-N-(pyren-1-yl)-[1,2,4]triazolo[1,5-a]pyrimidin-7-amine;
5-methyl-N-(3-hydroxynaphthalen-2-yl)-[1,2,4]triazolo[1,5-a]pyrimidin-7-amine,
5,6-dimethyl-N-(naphthalen-2-yl)-[1,2,4]triazolo[1,5-a]pyrimidin-7-amine;
N-(anthracen-2-yl)-5,6-dimethyl-[1,2,4]triazolo[1,5-a]pyrimidin-7-amine;
5-ethyl-N-(naphthalen-2-yl)-[1,2,4]triazolo[1,5-a]pyrimidin-7-amine; and
N-(anthracen-2-yl)-5-ethyl-[1,2,4]triazolo[1,5-a]pyrimidin-7-amine;
or a pharmaceutically acceptable salt, solvate, stereoisomer, tautomer, or prodrug thereof; and
(b) a pharmaceutically acceptable carrier.

20. The pharmaceutical composition of claim 19, wherein the compound is selected from the group consisting of:
5-methyl-N-(naphthalen-2-yl)-[1,2,4]triazolo[1,5-a]pyrimidin-7-amine;
5-methyl-N-(anthracen-2-yl)-[1,2,4]triazolo[1,5-a]pyrimidin-7-amine;
5-trifluoromethyl-N-(naphthalen-2-yl)-[1,2,4]triazolo[1,5-a]pyrimidin-7-amine;
5-methyl-N-(quinolin-6-yl)-[1,2,4]triazolo[1,5-a]pyrimidin-7-amine;
5-methyl-N-(4H-chromen-4-on-7-yl)-[1,2,4]triazolo[1,5-a]pyrimidin-7-amine;
5-methyl-N-(quinolin-3-yl)-[1,2,4]triazolo[1,5-a]pyrimidin-7-amine;
5-methyl-N-(pyren-1-yl)-[1,2,4]triazolo[1,5-a]pyrimidin-7-amine; and
5-methyl-N-(3-hydroxynaphthalen-2-yl)-[1,2,4]triazolo[1,5-a]pyrimidin-7-amine.

21. The pharmaceutical composition of claim 19, wherein the compound is 5-methyl-N-(naphthalen-2-yl)-[1,2,4]triazolo[1,5-a]pyrimidin-7-amine or a pharmaceutically acceptable salt, solvate, stereoisomer, tautomer, or prodrug thereof.

22. The method according to any one of claims 12 to 18, wherein the pharmaceutical composition comprises a compound selected from the group consisting of:
5-methyl-N-(naphthalen-2-yl)-[1,2,4]triazolo[1,5-a]pyrimidin-7-amine;
5-methyl-N-(anthracen-2-yl)-[1,2,4]triazolo[1,5-a]pyrimidin-7-amine;
5-trifluoromethyl-N-(naphthalen-2-yl)-[1,2,4]triazolo[1,5-a]pyrimidin-7-amine;
5-methyl-N-(quinolin-6-yl)-[1,2,4]triazolo[1,5-a]pyrimidin-7-amine;
5-methyl-N-(4H-chromen-4-on-7-yl)-[1,2,4]triazolo[1,5-a]pyrimidin-7-amine;
5-methyl-N-(quinolin-3-yl)-[1,2,4]triazolo[1,5-a]pyrimidin-7-amine;
5-methyl-N-(pyren-1-yl)-[1,2,4]triazolo[1,5-a]pyrimidin-7-amine;
5-methyl-N-(3-hydroxynaphthalen-2-yl)-[1,2,4]triazolo[1,5-a]pyrimidin-7-amine,
5,6-dimethyl-N-(naphthalen-2-yl)-[1,2,4]triazolo[1,5-a]pyrimidin-7-amine;
N-(anthracen-2-yl)-5,6-dimethyl-[1,2,4]triazolo[1,5-a]pyrimidin-7-amine;
5-ethyl-N-(naphthalen-2-yl)-[1,2,4]triazolo[1,5-a]pyrimidin-7-amine; and
N-(anthracen-2-yl)-5-ethyl-[1,2,4]triazolo[1,5-a]pyrimidin-7-amine;
or a pharmaceutically acceptable salt, solvate, stereoisomer, tautomer, or prodrug thereof.

23. The method according to claim 22, wherein the compound is selected from the group consisting of -p1 5-methyl-N-(naphthalen-2-yl)-[1,2,4]triazolo[1,5-a]pyrimidin-7-amine;
5-methyl-N-(anthracen-2-yl)-[1,2,4]triazolo[1,5-a]pyrimidin-7-amine;
5-trifluoromethyl-N-(naphthalen-2-yl)-[1,2,4]triazolo[1,5-a]pyrimidin-7-amine;
5-methyl-N-(quinolin-6-yl)-[1,2,4]triazolo[1,5-a]pyrimidin-7-amine;
5-methyl-N-(4H-chromen-4-on-7-yl)-[1,2,4]triazolo[1,5-a]pyrimidin-7-amine;
5-methyl-N-(quinolin-3-yl)-[1,2,4]triazolo[1,5-a]pyrimidin-7-amine;
5-methyl-N-(pyren-1-yl)-[1,2,4]triazolo[1,5-a]pyrimidin-7-amine; and
5-methyl-N-(3-hydroxynaphthalen-2-yl)-[1,2,4]triazolo[1,5-a]pyrimidin-7-amine.

24. The method according to any one of claims 12 to 18, wherein the pharmaceutical composition comprises 5-methyl-N-(naphthalen-2-yl)-[1,2,4]triazolo[1,5-a]pyrimidin-7-amine or a pharmaceutically acceptable salt, solvate, stereoisomer, tautomer, or prodrug thereof.
